(19) United States
(12) Patent Application Publication
(10) Pub. No.: US 2025/0148707 A1
(43) Pub. Date: May 8, 2025

(54) ARTIFICIAL INTELLIGENCE PHYSICS-BASED MODELING OF CARDIAC PARAMETERS

(71) Applicant: Delbeat, Inc., Berkeley, CA (US)

(72) Inventor: Mehrzad TARTIBI, Berkeley, CA (US)

(21) Appl. No.: 18/883,711

(22) Filed: Sep. 12, 2024

Related U.S. Application Data
(63) Continuation of application No. PCT/US2023/064547, filed on Mar. 16, 2023.
(60) Provisional application No. 63/320,965, filed on Mar. 17, 2022.

(51) Int. Cl. G06T 17/00; G06T 7/10; G16H 30/40

(52) U.S. Cl. CPC G06T 17/00; G06T 7/10; G16H 30/40; G06T 2207/30048; G06T 2210/41

(57) ABSTRACT

Described herein is software and computer implemented methods for modeling a heart of an individual where the model presents or displays one or more cardiac parameters of the individual. In some embodiments, a model of the heart generated by the software and computer implemented methods described herein is a three-dimensional representation of the heart of an individual that is displayable on a display of a computing device.

### CROSS-REFERENCE

This application is a continuation application of International Application PCT/US2023/064547, filed Mar. 16, 2023, which claims the benefit of U.S. Provisional Application No. 63/320,965, filed Mar. 17, 2022, which applications are hereby incorporated by reference in their entireties for all purposes.

### BACKGROUND

Many people suffer from cardiac disease. Various modalities are available for evaluating individuals' hearts for the purpose of diagnosing disease and assessing disease progression including but not limited to angiography, magnetic resonance imaging (MRI), ultrasound, and electrocardiogram (ECG). These modalities also sometimes include software used to record and display certain data.

### SUMMARY OF THE INVENTION

Described herein are methods and systems, and software, in combination with the systems, described elsewhere herein, that carry, execute, and/or preform the methods described elsewhere herein. In some cases, the methods, system, and/or software may analyze one or more images of a heart of an individual and generate and/or display (e.g., on a computing device display) one or more models of a heart of the individual that may comprise one or more cardiac parameters. The methods, systems, and/or software may provide a non-invasive modality for presenting data (displayed in a model of the heart) that typically must be collected through either or arduous or invasive testing. The methods, systems, and/or software may also provide an efficient modality for presenting multiple unique parameters that typically are collected from multiple different modalities. The methods, systems, and/or software may generate a model that is specific and/or tailored to an individual whose heart is imaged, rather than a generic model.

Traditionally, certain intensive clinical practices, imaging modalities, and invasive procedures are used as standard practice to understand a patient's specific cardiovascular anatomy and physiology. In general, these traditional techniques are time-consuming, invasive and/or painful for patients, and costly. These techniques are generally performed to understand parameters that describe a patient's heart including physiological, anatomical, and/or electrophysiological parameters of the patient's heart. In view of current methodologies and approaches, there exists an unmet need for a low-cost, non-invasive, and rapid method of determining traditional cardiac parameters. The methods, systems, and/or software described elsewhere herein address such an unmet need by non-invasively generating patient-specific cardiac models capable of determining, analyzing, and/or displaying one or more anatomical, physiological, and/or electrophysiological parameters of tissue in a non-invasive and/or minimally invasive manner. To further reduce the computational cost of such a platform, the methods, systems, and software described herein are configured to run on standard processing devices (e.g., personal computing device, laptop computing device, tablet, smart phone, virtual reality headset, etc.) without requiring the need of expensive super-computers or clustered computing devices that are traditionally used to generate models of patients' hearts when solving a brute force forward solution. Instead parallel processing architectures e.g., hyper threading or GPU based parallel processing are used to reduce processing times.

Aspects of the disclosure herein describe methods, systems, and/or software comprising algorithms which may determine patient cardiac parameters such as physiological, anatomical, and/or electrophysiological parameters. In some cases, the methods, systems, and/or software may be configured to analyze at least one image of a heart of a patient. In some embodiments, the at least one image of the heart of the patient comprises a regular MRI, time-resolved computed tomography angiography (CTA), 3D-echocardiogram, or any combination thereof images. In some embodiments, the methods, systems, and/or software analyze the at least one image and generate a model of the heart of the patient that can be viewed on an output display device e.g., screen of a computing device. In some embodiments, the output display device may comprise a user interface that comprises display information about one or more parameters of the heart. In some embodiments, the systems, and/or software execute the methods described elsewhere herein, using a typical processor of the system, e.g., a desktop or laptop computer or other computing device such as a smartphone, tablet, smartwatch, or any virtual reality device.

In some embodiments, described herein is a computer-implemented method for using a machine learning algorithm to model a heart of an individual, wherein the model comprises at least one of a functional feature and/or an electrophysiological feature of a heart, the method comprising: (a) receiving or obtaining, with an input software module, an image of the heart of the individual; (b) segmenting, with a segmenting software module, the image of the heart, thereby generating at least one image segment; (c) applying, with an analysis software module, a series of partial differential solvers to the at least one image segment, to solve a mathematical model, thereby generating an analysis result; and (d) generating, using a modeling software module, a model of at least one functional feature of the heart of the individual using the analysis result of (c). In some embodiments, the modeling software module generates a functional feature, an electrophysiological feature, or a combination thereof, of the heart of the individual using the analysis result of (c). In some embodiments, the series of partial differential solvers comprise finite element analysis. In some embodiments, the modeling software module comprises a machine learning algorithm. In some embodiments, the functional feature comprises a ventricular gauge pressure. In some embodiments, the functional feature comprises a wall thickness of a chamber of the heart. In some embodiments, the functional feature comprises a representation of a wall motion of at least a portion of the heart. In some embodiments, the functional feature comprises a representation of blood flow through the chambers of the heart. In some embodiments, the functional feature comprises a feature of a valve. In some embodiments, the functional feature comprises a perfusion feature, where the perfusion feature comprises a representation of myocardium perfusion through a tissue of the heart. In some embodiments, the electrophysiological feature comprises an electrical current flow through a tissue of the heart. In some embodiments, the functional feature comprises structural or dynamic characteristics of scar tissue of the heart. In some embodiments, the functional feature comprises spatial and/or temporal passive and/or active stress responses of a tissue of the heart. In some embodiments, the functional feature comprises spatial and/or temporal tissue passive and active material properties. In some embodiments, the functional feature comprises spatial and/or temporal tissue strain energy of a tissue of the heart. In some embodiments, the functional feature comprises spatial and/or temporal tissue mechanical efficiency of a tissue of the heart. In some embodiments, the electrophysiologic feature comprises spatial and/or temporal tissue activation time and strength of a tissue of the heart. In some embodiments, the electrophysiological feature comprises a current density map of a tissue of the heart. In some embodiments, the current density map comprises at least one current vector. In some embodiments, the individual suffers from heart failure, congestive heart failure, cardiac ischemia, heart valve disease, cardiomyopathy, heart attack, hypertrophic cardiomyopathy, pericarditis, pericardial effusion, heart murmurs, congenital heart disease, heart arrhythmias, coronary artery disease, or any combination thereof diseases. In some embodiments, the image of the heart is an MRI. In some embodiments, the image of the heart is an echocardiogram. In some embodiments, the echocardiogram comprises a 3D echocardiogram. In some embodiments, the segmenting software module comprises a level-set method, image processing techniques, and a machine learning algorithm trained using heart images from a population of individuals. In some embodiments, the at least one image segment shows at least a portion of a heart of the individual. In some embodiments, the analysis result comprises at least one equation that represents at least one factor associated with the image of the heart. In some embodiments, the machine learning algorithm is trained using parameter data from a population of individuals. In some embodiments, the method further comprises displaying the model on a display of a computing device.

In some embodiments, described herein is a non-transitory computer readable medium that includes software, where the software uses machine learning to model a heart of an individual. In some embodiments, the model of the heart generated may comprise at least one of a functional feature. In some cases the software may causes a processor of a computer system to: (a) receive an image of the heart of the individual; (b) segment the image of the heart, thereby generating at least one image segment; (c) apply an equation solver to the at least one image segment, thereby generating an analysis result; and (d) generate, using partial differential equation solver and/or a machine learning algorithm, a model of at least one functional feature and/or at least one electrophysiological feature of the heart of the individual using the analysis result of (c).

In some embodiments, a segmentation module configured to segment an image of the heart comprises a machine learning algorithm. In some cases, the machine learning algorithm may improve the accuracy of the segmentation algorithm by at least about 80%, at least about 82%, at least about 84%, at least about 86%, at least about 88%, at least about 90%, at least about 92%, at least about 94%, at least about 96%, at least about 98%, or at least about 99%.

In some embodiments, the equation solver comprises a partial differential equation solver. In some embodiments, the partial differential equation solver uses finite element analysis. In some embodiments, the model generated comprises at least one functional feature at least one electrophysiological feature, or a combination thereof, of a heart of the individual. In some embodiments, the functional feature comprises a ventricular and/or atrial gauge pressure. In some embodiments, the functional feature comprises a wall thickness of one or more chambers of the heart. In some embodiments, the functional feature comprises a representation of a wall motion of at least a portion of the heart. In some embodiments, the functional feature comprises a representation of blood flow through one or more chambers of the heart. In some embodiments, the functional feature comprises a feature of a valve of the heart. In some embodiments, the electrophysiological feature comprises a representation of myocardium perfusion through a tissue of the heart. In some embodiments, the functional feature comprises structural and/or dynamic characteristics of scar tissue of the heart. In some embodiments, the functional feature comprises tissue perfusion of the heart. In some embodiments, the functional feature comprises spatial and/or temporal passive and/or active stress response of a tissue of the heart. In some embodiments, the functional feature comprises spatial and/or temporal tissue passive and/or active material properties. In some embodiments, the functional feature comprises spatial and/or temporal tissue strain energy of a tissue of the heart. In some embodiments, the functional feature comprises spatial and/or temporal tissue mechanical efficiency of a tissue of the heart. In some embodiments, the electrophysiologic feature comprises spatial and/or temporal tissue activation time and strength of a tissue of the heart. In some embodiments, the model comprises a current density map of a tissue of the heart. In some embodiments, the current density map includes at least one current vector. In some embodiments, the individual suffers from heart failure, congestive heart failure, cardiac ischemia, heart valve disease, cardiomyopathy, heart attack, hypertrophic cardiomyopathy, pericarditis, pericardial effusion, heart murmurs, congenital heart disease, heart arrhythmias, coronary artery disease, or any combination thereof diseases. In some embodiments, the image of the heart is an MRI image. In some embodiments, the image of the heart is an echocardiogram image. In some embodiments, the image of the heart is a time-resolved CTA image. In some embodiments, the echocardiogram image is generated by 3Dechocardiogram. In some embodiments, the MRI image comprises a 4D MRI image or image dataset. In some embodiments, the image comprises a computed tomography angiography (CTA) image. In some cases, the computed tomography angiography image comprises a 4D computed tomography angiography image or image dataset. In some embodiments, the at least one image segment shows at least a portion of the heart of the individual. In some embodiments, the analysis result comprises at least one equation that represents at least one factor associated with the image of the heart. In some embodiments, the machine learning algorithm is trained using parameter data from a population of individuals. In some embodiments, the method further comprises displaying the model on a display of a computing device. In some cases, the segmentation of an image of the heart comprises a level-set method or other image processing methodologies such as cross-correlation, convolution, clustering, applied graph theory, or any combination thereof methods.

Aspects of the disclosure describe a computer-implemented method for generating a model of a heart of an individual, the method comprising: (a) receiving or obtaining, with an input software module, an image of the heart of the individual; (b) segmenting, with a segmenting software module, the image of the heart, thereby generating at least one image segment; (c) applying, using an analysis software module, one or more differential equations to the at least one image segment, thereby generating at least one analysis result; and (d) generating, using a modeling software module, the model of the heart of the individual using the analysis result of (c). In some embodiments, the analysis result comprises at least one functional feature, at least one electrophysiological feature, or any combination thereof features. In some embodiments, the functional feature comprises a ventricular gauge pressure of a right and left ventricle. In some embodiments, the functional feature comprises a wall thickness of a chamber of the heart. In some embodiments, the functional feature comprises a wall motion of at least a portion of the heart. In some embodiments, the functional feature comprises blood flow through the chambers of the heart. In some embodiments, the functional feature comprises a feature of a valve. In some embodiments, the electrophysiological feature comprises an electrical property of a tissue of the heart. In some embodiments, the electrical property comprises an activation map, voltage map, or any combination thereof. In some embodiments, the electrical property comprises at least one current vector. In some embodiments, the individual suffers from or is suspected of suffering from heart failure. In some embodiments, the image of the heart is an MRI image. In some embodiments, the image of the heart is an echocardiogram image. In some embodiments, the echocardiogram comprises a 3D echocardiogram. In some embodiments, the segmenting software module comprises a machine learning algorithm, artificial intelligence, or a combination thereof, trained using one or more heart images from a population of individuals. In some embodiments, the at least one image segment shows at least a portion of a heart of the individual. In some embodiments, the machine learning algorithm, artificial intelligence, or a combination thereof, is trained with segmentation data from a population of individuals. In some embodiments, the computer-implemented method further comprises (e) displaying the model on a display of a computing device. In some embodiments, the image of the heart may comprise one or more images of the heart, images of an imaging study, or any combination thereof. In some embodiments, the one or more differential equations comprise one or more partial, ordinary, or any combination thereof differential equations. In some embodiments, the one or more differential equations comprise finite element analysis equations. In some embodiments, the computer-implemented method further comprises generating a therapeutic guiding parameter model with the analysis result of (c). In some embodiments, the computer-implemented method further comprises generating a growth remodeling parameter model with the analysis results of (c) longitudinally over a period of time.

Aspects of the disclosure describe a non-transitory computer readable medium that includes software that uses artificial intelligence to model a heart of an individual, wherein the software causes a processor to: (a) receive or obtain an image of the heart of an individual; (b) segment the image of the heart, thereby generating at least one image segment; (c) apply a series of numerical techniques to the at least one image segment, thereby generating an analysis result; and (d) generate, a model with at least one functional feature, at least one electrophysiological feature, or a combination thereof, of the heart of the individual using the analysis result of (c). In some embodiments, the functional feature comprises a ventricular gauge pressure. In some embodiments, the functional feature comprises a wall thickness of a chamber of the heart. In some embodiments, the functional feature comprises wall kinematics or kinetics of at least a portion of the heart. In some embodiments, the functional feature comprises blood flow through one or more chambers of the heart. In some embodiments, the functional feature comprises a feature of a valve. In some embodiments, the electrophysiological feature comprises an electrical property of a tissue of the heart. In some embodiments, the electrical property comprises an activation map, voltage map, or any combination thereof. In some embodiments, the electrical property comprises at least one current vector. In some embodiments, the individual suffers from heart failure. In some embodiments, the image of the heart is an MRI image. In some embodiments, the MRI comprises CINE MRI, MRI based techniques, or any combination thereof. In some embodiments, the MRI based techniques comprise DENSE, tag-MR, SPAMM, or any combination thereof. In some embodiments, the image of the heart is an echocardiogram image. In some embodiments, the echocardiogram comprises a 3D echocardiogram. In some embodiments, the at least one image segment shows at least a portion of a heart of the individual. In some embodiments, the numerical techniques comprise at least one equation that represents at least one functional feature, at least one electrophysiologic feature, or a combination thereof, associated with the image of the heart. In some embodiments, the software comprises a segmentation algorithm that segments the image of the heart. In some embodiments, the segmentation algorithm comprises a machine learning algorithm, artificial intelligence, or a combination thereof. In some embodiments, the machine learning algorithm comprises one or more machine learning algorithms, an ensemble of machine learning algorithms, or any combination thereof. In some embodiments, the machine learning algorithm, artificial intelligence, or a combination thereof, is trained with segmentation data from a population of individuals. In some embodiments, the software further causes the processor to (e) display the model on a display of a computing device, virtual 3D device, or virtual 4D device. In some embodiments, the numerical technique comprises finite element analysis technique. In some embodiments, the display of the computing device comprises a screen of the computing device, augmented reality display, virtual reality display, or any combination thereof displays. In some embodiments, the software further causes the processor to generate a model comprising a therapeutic guiding parameter determined by the at least one functional feature, the at least one electrophysiological feature, or a combination thereof, of the heart of the individual. In some embodiments, the software further causes the processor to generate a model comprising a growth remodeling parameter determined by the at least one functional feature, the at least one electrophysiological feature, or a combination thereof, of the heart of the individual.

Aspects of the disclosure describe a method for determining a material property of a region of a heart of an individual, the method comprising: (a) receiving or obtaining an image of the heart of an individual; (b) segmenting the image of the heart, thereby generating at least one image segment; (c) applying one or more differential equations to the at least one image segment, thereby generating at least one analysis result, wherein the at least one analysis result comprises a functional parameter, an electrophysiological parameter, or a combination thereof; and (d) determining a material property of at least one region of the heart of the individual by the at least one analysis result. In some embodiments, an analysis software module applies the one or more differential equations to the at least one image segment. In some embodiments, the image is received or obtained by an image input software module. In some embodiments, a segmenting software module segments the image of the heart. In some embodiments, the image comprises one or more time-resolved series images. In some embodiments, the functional feature comprises a ventricular gauge pressure of a right or left ventricle. In some embodiments, the functional feature comprises blood perfusion of a tissue of the heart. In some embodiments, the functional feature comprises a wall thickness of one or more chambers of the heart. In some embodiments, the functional feature comprises wall motion of at least a portion of the heart. In some embodiments, the functional feature comprises blood flow through one or more chambers of the heart. In some embodiments, the functional feature comprises a feature of a valve. In some embodiments, the electrophysiological feature comprises an electrical property of a tissue of the heart. In some embodiments, the electrical property of the tissue comprises an activation map, voltage map, or a combination thereof. In some embodiments, the electrical property comprises at least one current vector. In some embodiments, the individual suffers from or is suspected of suffering from heart failure. In some embodiments, the image of the heart is an MRI, 4D MRI, time-resolved computed tomography angiography (CTA), 3D echocardiogram, computed tomography (CT), 3D CT, 4D CT, or any combination thereof image. In some embodiments, segmenting comprises thresholding segmentation, fast marching level-set segmentation, hands-free segmentation, or any combination thereof. In some embodiments, the segmentation software module comprises a machine learning algorithm, artificial intelligence, or any combination thereof, configured to segment the image of the heart, wherein the machine learning algorithm, artificial intelligence, or a combination thereof, is trained with an output of the thresholding segmentation, fast marching level-set segmentation, hands-free segmentation, or any combination thereof. In some embodiments, the at least one image segment shows at least a portion of a heart of the individual. In some embodiments, the machine learning algorithm, artificial intelligence, or a combination thereof, is trained with segmentation data from a population of individuals. In some embodiments, the method further comprises (e) displaying a model of the heart of the individual, wherein the model is generated with at least the at least one analysis result and the material property of at least one region of the heart. In some embodiments, the model is displayed on a display of a computing device. In some embodiments, the image of the heart may comprise one or more images of the heart, images of an imaging study, or any combination thereof. In some embodiments, the one or more differential equations comprise one or more partial, ordinary, or any combination thereof differential equations. In some embodiments, the one or more differential equations comprise finite element analysis equations. In some embodiments, the method further comprises generating a therapeutic guiding parameter model with the analysis result of (c). In some embodiments, the method further comprises generating a growth remodeling parameter model with the analysis results of (c) longitudinally over a period of time. In some embodiments, steps (b)-(d) are completed in up to about 1 hour.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic representation of exemplary inputs and outputs of the software, as described in some embodiments herein.

FIG. 2 shows a schematic representation of an exemplary workflow that utilizes the software, as described in some embodiments herein.

FIGS. 3A-3C show an exemplary operational workflow and relationship of the various software modules configured to compute and represent data model representations of clinical outcomes and classifications, as described in some embodiments herein.

### DETAILED DESCRIPTION OF THE INVENTION

Cardiac imaging technologies have improved significantly over the past two decades. The current cardiac imaging modalities, such as MRI, ultrasound, and computed tomography (CT), provide dynamic information of blood flow and the motion of heart tissue. However, such imaging modalities are limited in that they do not measure functional tissue information from their structural imaging data output. Currently, functional imaging of organ systems such as the heart are conducted by expensive, ionizing radiation and/or invasive imaging modalities e.g., SPECT, PET, and/or PET-MR. There currently does not exist a cost-effective, minimally invasive platform to determine heart physiologic and anatomical information in an integrated manner. Moreover, the current methods, systems, and/or software configured to determine heart physiologic and anatomical information require extensive computational resources (e.g., a computer cluster with more than 16 processor cores) and substantial amount of processing time (e.g., more than 8 hours and up to a week) to produce the physiologic and anatomical analysis results that is impractical and unmanageable when utilized in a real-world large patient populations.

Described herein are methods, system, software, and computer-implemented methods for modeling and analyzing the motion of one or more regions of patients' heart wall, heart chambers, heart tissue, or any combination thereof, determined from dynamic (temporal and/or spatial) structural clinical cardiac imaging modalities described above (e.g., MRI, computed tomography, computed tomography angiography (CTA), time-resolved CTA, echocardiogram, etc.). In some instances, the methods, system, software and computer-implemented methods described herein, may utilize non-ionizing imaging data, e.g., structural MRI, ultrasound, etc., to determine functional and/or electrophysiologic information of a patient's heart (e.g., an electrophysiology current density map and/or metabolic energetics distribution). In some instances, the software and computer-implement methods described elsewhere herein may be categorized as structural and functional imaging techniques (FIT). Some of unexpected advantages of the software and computer-implement methods over traditional techniques, described elsewhere herein, may comprise: (1) reduced harmful exposure to ionizing radiation and isotopes; (2) faster processing times compared to traditional FITs; (3) additional functional measures of the heart, such as local energy density, (4) reduced cost in comparison to traditional FITs; (5) comprehensive three-dimensional datasets of electrophysical measurements compared to traditional two-dimensional surface electrophysiology measures; and (6) a higher spatial resolution compared to traditional FITs.

Described herein are methods, systems, software and/or computer implemented methods for modeling a heart of an individual where the model presents or displays one or more cardiac parameters of the individual. In some embodiments, a model of the heart generated by the methods, systems, software and/or computer implemented methods described herein, may generate a three-dimensional representation of the heart of a subject that may be displayed on a screen or display of a computing device. In some cases the subject is human, a non-human mammal, non-human non-mammal, or any combination thereof. As described above, the methods, systems, software, and/or computer implemented methods are beneficial in at least that they provide a non-invasive, quick, efficient (in terms of computing power required) and cost-effective means of obtaining a variety of different types of personalized cardiac data. In contrast, traditional techniques are invasive (or otherwise burdensome), time consuming, require far greater computing power, and are far more expensive. The software and computer implemented methods described herein are also beneficial since they provide, in a single workflow, data and/or analysis results (e.g., the functional and/or electrophysiologic features of a heart) that would otherwise require multiple different imaging modalities, procedures, tests, and/or workflows.

The methods, systems, software and/or computer-implemented methods, described herein, may use the full-field tissue displacement, determined from images of patients' hearts. In some cases the methods, systems, software and/or computer implemented methods may use direct displacement velocity measure MRI based techniques e.g., DENSE, tag-MR, SPAMM, or any combination thereof as inputs. These full-field tissue displacements are calculated using only multiple coupled physics-based algorithms, which do not require mathematical assumptions. Such mathematical assumptions may comprise assumptions associated with material mechanical constitutive laws or assumptions associated with the myocardial electrical tissue network and tissue conductance tensor.

The methods, systems, software and/or computer-implemented methods provide better than expected results of anatomical, physiologic, and/or electrophysiological features of a tissue of a heart for number of reasons, including that: (a) full-field tissue displacement as a function of time are measured directly from the structural images that are otherwise not measured using typical clinical MR-based imaging, (b) there is no reliance on the aforementioned assumptions, (c) local tissue characteristics (and thus, the passive and/or active tissue health) are determined without ex vivo testing of the tissue, (d) the myocardium electrical tissue network geometry does not need to be known to generate the models described herein, (e) the software is computationally inexpensive, as it does not require computer clusters-after collecting sufficient data points, the instant methods, systems, and/or software use specific image processing, deep learning and artificial intelligence (AI) algorithms to provide the segmentation and analysis results after uploading the cardiac image files to the cloud, and (f) the software may predict patients' mechanical and electrical response to particular treatments, such as ACORN (cardiac restraint device), MitroClip, ventricle assist device, gel-injection therapy, cardiac resynchronization therapy, or any combination thereof treatments.

### Overview

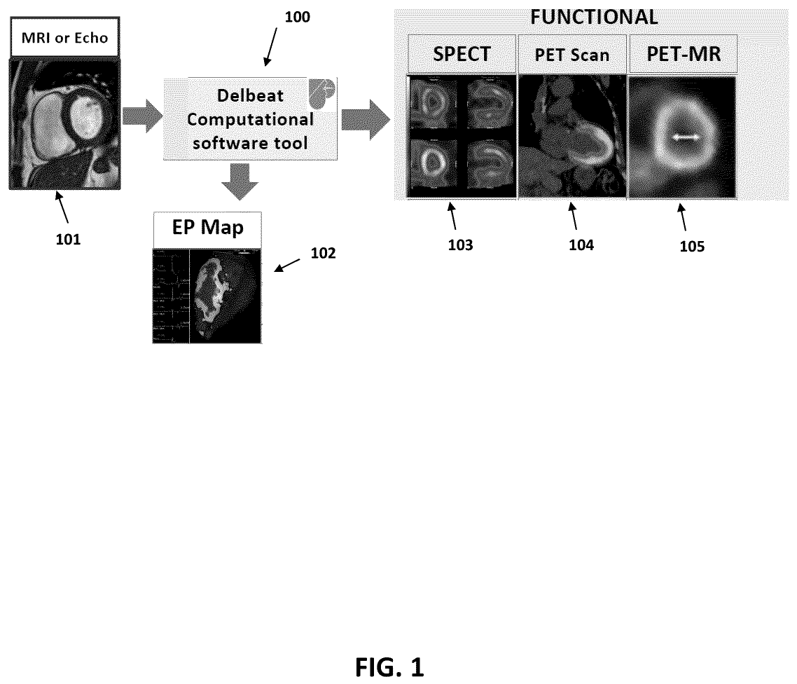

FIG. 1 shows a flow diagram representation of the software 100, described herein. As shown, an image of the heart 101 may be provided as an input for software 100, which may output a model that provides or displays the information shown in a traditional electrophysiologic map 102, a single-photon emission computed tomography (SPECT) scan 103, a positron emission tomography (PET) scan 104, a positron emission combined magnetic resonance tomography (PET-MR) scan 105, a number of additional cardiac measures (e.g., functional, anatomical, and/or electrophysiologic), or any combination thereof.

In some embodiments, the image of the heart 101 may comprises a magnetic resonance imaging (MRI) scan, echocardiogram, computed tomography angiography (CTA), time-resolved CTA, or any combination thereof image of the heart. In some embodiments, the image of the heart 101 may comprises a cardiac CINE MRI image. In some embodiments, an echocardiogram of the heart may comprise at least a one-dimensional, at least a two-dimensional, at least a three-dimensional, or at least a four-dimensional echocardiogram. It should, however, be understood that other types of images that show the anatomy of the heart, understood by one of ordinary skill in the art, may be suitable for use with the software and methods described herein in, non-limiting examples of which include: computed tomography (CT), CT-angiography, two-dimensional x-ray, or any combination thereof.

In some embodiments, the output of the software, systems, and methods may provide a noninvasive measurement of the ventricular cavity gauge pressure. In some cases the methods, systems, and/or software, described elsewhere herein, may comprise a non-invasive platform that provides better than expected results in view of an invasive counterpart. The disclosure provided herein, in some embodiments, describes a non-invasive software configured to measure the left and/or right ventricle pressures of subjects' hearts by analyzing images of subjects' hearts collected non-invasively, described elsewhere herein. In contrast, invasive approaches measure cavity pressures directly by: (1) transcatheter pressure transducer(s) placed subcutaneously inside the chamber cavity, or (2) microelectromechanical systems (MEMS) placed in the outflow tracks wall subcutaneously for ICU patients for continuous beat-by-beat blood pressure measurement.

Additionally, the software, systems, and/or methods, described herein, are not limited in particular breadth of simultaneous measurements that may be made and analyzed in comparison to prior devices, systems, and methodologies. For example, not only is a transcatheter pressure transducer an invasive medical device it would be limited in its functionality of solely measuring chamber pressure. Whereas the software, systems, and/or methods may non-invasively determine chamber pressure but may also simultaneously measure tissue mechanical active and/or passive properties on three-dimensional macro and/or micro tissue and/or organ scale.

The software, systems, and/or methods may be configured to measure tissue stored stress in a non-nondestructive manner. Other potential methods understood by one of ordinary skill in the art may approximate the residual stresses in a heart's vertical wall destructively through ex-vivo experiments. Knowledge of stored stress provides actionable clinical insight to diastolic heart failure (in some cases heart failure with preserved ejection fraction, HFpEF) patients. Furthermore the software, systems, and/or methods may directly measure active myocardium contraction stress. One of ordinary skill in the art would appreciate that the software, methods, and/or systems provide a solution to the unmet need of directly and non-invasively measuring the local active myocardium contraction stress and/or energy production density.

In some embodiments, the software, system, and/or methods may determine in-vivo spatial and temporal measurements of the full strain tensor field (e.g., up to six strains) using one or more images of subjects' hearts, for example, using one or more CINE MRI images of a heart.

In some cases, the one or more images may comprise about 1 image to about 10,000 images. In some cases, the one or more images may comprise about 1 image to about 5 images, about 1 image to about 10 images, about 1 image to about 25 images, about 1 image to about 50 images, about 1 image to about 100 images, about 1 image to about 150 images, about 1 image to about 500 images, about 1 image to about 1,000 images, about 1 image to about 10,000 images, about 5 images to about 10 images, about 5 images to about 25 images, about 5 images to about 50 images, about 5 images to about 100 images, about 5 images to about 150 images, about 5 images to about 500 images, about 5 images to about 1,000 images, about 5 images to about 10,000 images, about 10 images to about 25 images, about 10 images to about 50 images, about 10 images to about 100 images, about 10 images to about 150 images, about 10 images to about 500 images, about 10 images to about 1,000 images, about 10 images to about 10,000 images, about 25 images to about 50 images, about 25 images to about 100 images, about 25 images to about 150 images, about 25 images to about 500 images, about 25 images to about 1,000 images, about 25 images to about 10,000 images, about 50 images to about 100 images, about 50 images to about 150 images, about 50 images to about 500 images, about 50 images to about 1,000 images, about 50 images to about 10,000 images, about 100 images to about 150 images, about 100 images to about 500 images, about 100 images to about 1,000 images, about 100 images to about 10,000 images, about 150 images to about 500 images, about 150 images to about 1,000 images, about 150 images to about 10,000 images, about 500 images to about 1,000 images, about 500 images to about 10,000 images, or about 1,000 images to about 10,000 images. In some cases, the one or more images may comprise about 1 image, about 5 images, about 10 images, about 25 images, about 50 images, about 100 images, about 150 images, about 500 images, about 1,000 images, or about 10,000 images. In some cases, the one or more images may comprise at least about 1 image, about 5 images, about 10 images, about 25 images, about 50 images, about 100 images, about 150 images, about 500 images, or about 1,000 images. In some cases, the one or more images may comprise at most about 5 images, about 10 images, about 25 images, about 50 images, about 100 images, about 150 images, about 500 images, about 1,000 images, or about 10,000 images.

In some cases, the methods, systems, and/or software may implement tissue displacement tracking to compute an entire strain field. In some instances, the entire strain field may be used measure of akinetic properties of a tissue of the heart.

In some embodiments, the software, systems, and/or methods described herein may determine in-vivo spatial measurements of stored energy, i.e., stored stress and/or residual stress, of a subject's heart ventricle walls. In some cases, stored strain and stress may be directly proportional to the heart myocardium tissue health.

In some embodiments, the software, systems, and/or methods may determine the spatial and/or temporal force, stress, and/or energy fields generated by heart myocardium contraction(s). In some embodiments, models generated by the methods, system, and/or software may present or display the coupling of electrical stimuli and muscle contraction throughout a full cardiac cycle.

In some embodiments, the software, systems, and/or methods may determine an in-vivo measurement of the spatial and/or temporal electrical potential difference field non-invasively, thereby leading to a measurement of a tissue conductance tensor field. In some embodiments, the software, systems, and/or methods may determine myocardium in vivo electrical activities as a function of time and three-dimensional space, i.e., a four-dimensional measurement including both time and three-dimensional space.

Additional details regarding the software, systems, and/or methods may be found in the following sections.

### Software

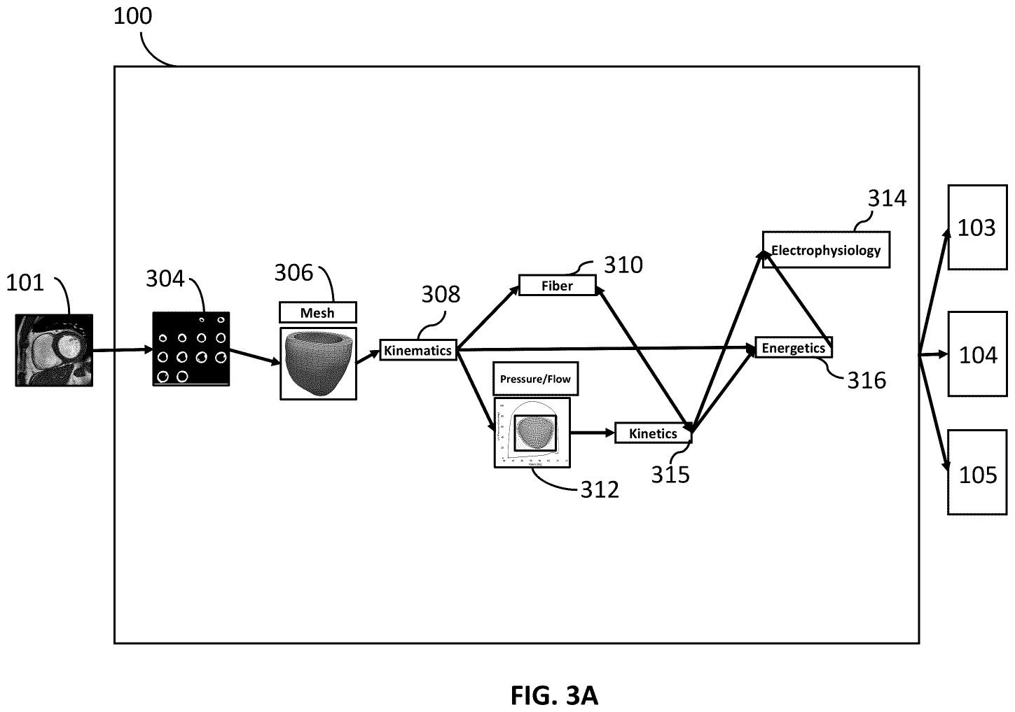

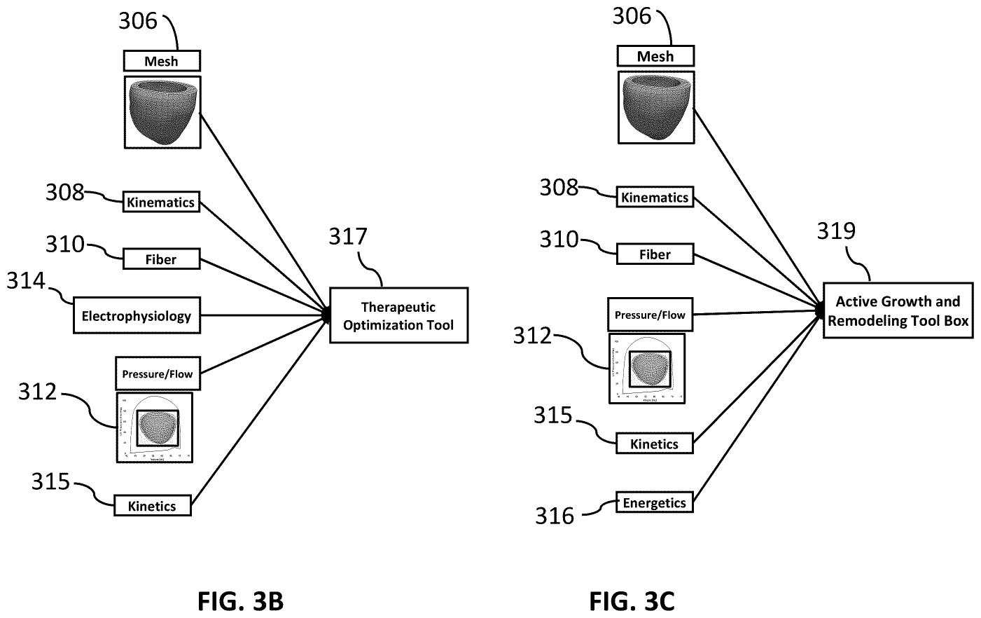

The software 100, described herein, may comprise a sequence of computer-readable instructions, executable by one or more processor(s) of a computing device's processor e.g., central processing unit (CPU), written to perform a specified task. Computer-readable instructions can be implemented as software modules, such as functions, objects, Application Programming Interfaces (APIs), computing data structures, or any combination thereof, that perform particular tasks or implement particular abstract data types, as seen in FIGS. 3A-3C. In light of the disclosure provided herein, those of skill in the art will recognize that software can be written in various versions of various languages.

The functionality of the computer-readable instructions can be combined or distributed as desired in various embodiments. In some embodiments, the software described herein may comprise one sequence of instructions. In some embodiments, the software described herein may comprise a plurality of sequences of instructions. In some embodiments, the software described herein may comprise at least one sequence of instructions. In some embodiments, the software described herein is provided from one location e.g., the software may be loaded into a stand-alone computing device. In other embodiments, the software described herein may be accessed from a plurality of locations e.g., where the software is located on a cloud-computing or other remote server platform and may be accessed from one or more user locations at a computer via the server. In some embodiments, the software described herein may comprise one or more software modules. In various embodiments, the software described herein may comprise, in part or in whole, one or more web applications, one or more mobile applications, one or more standalone applications, one or more web browser plug-ins, extensions, add-ins, add-ons, or any combination thereof.

In some embodiments, the software 100 may comprise one or more software modules and/or utilize one or more software modules, as shown in FIGS. 3A-3C. The software module(s) described herein may be implemented in a multitude of ways. In some embodiments, a software module may comprise a file, a section of code, a programming object, a programming structure, or any combination thereof. In some cases, a software module may comprise a plurality of files, a plurality of sections of code, a plurality of programming objects, a plurality of programming structures, or any combination thereof. In some instances, the one or more software modules may comprise, by way of non-limiting examples, a web application, a mobile application, and/or a standalone application. In some embodiments, multiple software modules may be a single computer program or application. In some cases, the one or more software modules may be located in a plurality of computer programs and/or applications. In some cases, software modules may be hosted on one machine. In some instances, software modules may be hosted on a plurality of machines. In some cases, software modules may be hosted on a distributed computing platform e.g., a cloud computing platform. In some embodiments, software modules may be hosted on one or more machines in one location. In some cases, software modules may be hosted on one or more machines in more than one location. It should therefore be understood that the software described herein may be run on a first computing device and access a software module on a second computing device or stored in the cloud. For example, when software described herein is said to utilize a software module, the software module may be on the same computing device as the software utilizing it or on a separate computing device or the cloud.

Non-limiting examples of types of modules that can be found in some embodiments of the innovative software described herein may include: input software modules, segmenting software modules, analysis software modules, modeling software modules, or any combination thereof.

### Input Software Module

In some embodiments, an input software module may comprise a component of and/or is utilized by software 100 to generate one or more anatomical, physiological, and/or electrophysiological features of a tissue of a heart. The input software module may be configured to receive and/or process data 101 transmitted to a computing device. In some instances, data received by the input software module may be associated with and/or represent a heart of an individual. In some cases, the data that is received by the input software module may comprise an image of a heart or a portion of a heart. In some instances, the data received by the input software module may comprise non-static, i.e., dynamic time-varying images, such as, for example, images produced by imaging modalities that show cardiac motion and/or blood flow. Non-limiting examples of modalities that show cardiac motion may comprise echocardiogram, MRI, computed tomography angiography, time-resolved CTA, or any combination thereof. In some cases, echocardiogram may comprise at least a one-dimensional, at least a two-dimensional, at least a three-dimension, or at least a four-dimension echocardiogram. In some instances, the MRI modality may comprise a CINE MRI. In some instances, MRI may comprise 3D MRI or 4D MRI. In some cases, the modality that shows cardiac motion may comprise DENSE, Tag-MR, SPAMM, or any combination thereof. As used herein an “image” may comprise a single image or a collection of images together (e.g., a video or motion capture). As used herein “Static” refers to images that do not capture cardiac motion (i.e., kinetics). As used herein “Non-static” refers to images that show cardiac motion (i.e., kinetics).

Data received by the input software module, in some embodiments, may comprise non-image data specific to the individual such as electro-cardiogram (ECG), electronic medical record (EMR) data, and/or subject and/or patient demographic data. In some cases, demographic data comprises gender, age, ethnicity, city and state of residency, or any combination thereof of subject data. In some instances, EMR data may comprise blood pressure, cholesterol, antibody panel readings, white blood cell count, troponin levels, blood sugar levels, hematocrit, medical history of past disease or conditions of the individual, whether or not the individual is a smoker or drinks alcohol and to what frequency the individual smokes or consumes alcoholic beverages, or any combination thereof data.

In some cases, non-limiting examples of data received by the input software module may comprise: demographic data, medical record data (including electronic medical record data) ECG data, echocardiogram data including two-dimensional, three-dimensional, and/or four-dimensional echocardiogram data, MRI data including CINE MRI, CT data, CT angiogram data, time-resolved CTA data, X-ray data, angiogram data, or any combination thereof. Data that is received may be complete or partial with respect to the data itself. In some cases, data received may be associated with, relates to, and/or shows the heart of an individual who is being evaluated for cardiac disease, cardiac malformation and/or suspected of having cardiac disease. In some cases, data received may comprise a retrospective dataset of individuals of a population that at the time the data was recorded have a cardiac disease or were not suspected of having a cardiac disease.

Data may be received by the software through any known means of communication including through wired or wireless communication means with another computing device, server, cloud, device capable of transmitting data to the computing device that is running the input software module, or any combination thereof.

In some cases, data received by the input software module may also be processed by the input software module. For example, in some cases, data received may be identified (e.g., with respect to content or type of data) by the input software module and assigned a value based on its identification which further determines how the received data may be handled by the software algorithm. For example, an ECG that is received by the input software module, in some embodiments, may be identified as an ECG rather than an image of the heart and then may be transmitted to a different software module compared to a software module that would process an image of a heart. In some cases, static images of the heart may be identified and transmitted to a different software module than non-static images of the heart. In some instances, the input software module may clean the received data with respect to, for example, associated metadata and labels. In some cases, the input software module may clean the received data to remove patient and/or individual identifiable data (e.g., individual's name, address, etc.). In some cases, cleaning may comprise denoising the data to remove image artifact noise and to increase signal to noise ratios of relevant signals and/or regions of the data to be analyzed.

After receiving data, the software may further process the received data. In some cases, the input software module may transmit the data (including pre-and post-processed data) to another module within the software such as, for example, a segmenting software module, an analysis software module, and/or a modeling software module.

### Segmenting Software Module

In some embodiments, the software described herein may comprise a segmenting software module 304, as seen in FIG. 3A. In some cases, the segmenting software module 304 may be configured to segment one or more images of a heart that is received. In some cases, the segmentation software module may remove and/or reduce motion artifact induced during initial acquisition of the one or more images 101 inputted into the method, system, and/or software. In some instances, the one or more images may comprise static and/or non-static images. In some instances, segmenting may comprise the process of segmenting data found in an image into different data sub-types i.e., data collections. For example, a segmenting software module may be configured to separate data of an image into a first component determined to be signal and second component that are determined to be background (i.e., data that is not part of the signal). In some cases, the first component of the data determined to be signal by the software may comprise data that the software may further analyze to arrive at a diagnostic indicator, a functional parameter, a physiologic parameter, an electrophysiologic parameter, or any combination thereof. In some cases, the second component of the data determined to be background may be subtracted from the original data. For example, in some cases, the segmenting software module may identify a segment a heart within an image and separate the data associated with the heart from background elements in the image. In some instances, segmentation conducted by the segmentation software module, may be applied to both static and non-static image components. Segmentation may be applied to different anatomical components of a heart. In some cases, the different anatomical components of a heart may comprise the aorta, atria, tri-leaflet valves, SA-node, purkinje fibers, bi-leaflet valves, ventricles, or any combination thereof components. For example, image data may be segmented to separate (or isolate) the segment of the image representing the aorta out of an image of a heart.

In some embodiments, the segmenting software module may be configured to remove environment or patient induced motion artifact present in a dynamic volumetric images of a patient and/or subject's internal organ(s). In some cases, the induced motion artifact may be caused by a patient breathing and/or macro-scale body movements while one or more volumetric and/or temporal volumetric images of a patient are acquired. In some cases, environmental induced motion artifact may comprise motion of the static surface upon which the subject and/or patient is resting on during image acquisition. In some instances, environmental induced motion artifact may comprise motion of the image acquisition system with respect to the patient or subject imaged.

In some cases, the segmenting software module may remove environment or patient induced motion artifact present in the imaging dataset by spatially tracking and re-aligning an identified surface of one or more two-dimensional cross-sectional images. In some instances, spatially tracking and re-aligning a time-series of two-dimensional images will greatly improve the sensitivity, accuracy, and/or analysis of downstream one or more software modules (e.g., 306, 308, 310, 312, 314, 315, 316, or any combination thereof). In some cases the two-dimensional cross-sectional images may comprise images generated by volumetric imaging modalities, described elsewhere herein, e.g., MRI, CT, time resolved CTA, echocardiogram, or any combination thereof. In some cases, the volumetric images may represent a time series of volumetric images e.g., a series of cross-sectional images (i.e., a volumetric dataset) of an organ(s) system of a subject or patient over time. In some cases, the one or more two-dimensional cross-sectional images may be re-aligned through methods of cross-correlation and/or feature co-registration. In some cases, the segmenting software module may conduct at least two sets of two-dimensional cross-sectional image re-alignment, where the two sets of two-dimensional cross-sectional images comprise orthogonal imaging planes. For example if an X, Y, and Z three-dimensional space coordinate system would be imposed upon a subject at a given orientation of the subject, one set of the two-dimensional cross-sectional images may comprise images along a plane of the X and Y axis whereas the other two-dimensional cross-sectional image may comprise images along a plane of Y-Z. In some embodiments, the orthogonal two-dimensional cross-sectional images may comprise images along any of the following image planes: X-Y plane, X-Z plane, or Y-Z plane.

In some embodiments, the segmenting software module may segment imaging datasets using thresholding segmentation, fast marching level-set segmentation, hands-free segmentation, or any combination thereof.

In some embodiments, the segmenting software module may be configured to utilize one or more artificial intelligence and/or machine learning algorithms. The one or more artificial intelligence or machine learning algorithm may comprise a neural network. In some instances, the one or more machine learning and/or the artificial intelligence algorithms may utilize computer vision to analyze the received one or more images. In some embodiments, the machine learning algorithm and/or the artificial intelligence may comprise an algorithm that is initially trained on a population of subjects' segmented data, segmented using any of the aforementioned segmentation techniques, described elsewhere herein. In some cases, the population data may comprise image and non-image data. In some embodiments, the population data may comprise both normal (i.e., data of individuals confirmed to not have a cardiac disease or condition) and abnormal data (i.e., data of individuals confirmed to have a cardiac disease or pre-disease states).

In some cases, the segmenting software module may output one or more datasets that have been separated from other data within an image, described elsewhere herein. For example, a dataset output may comprise a heart of an individual (either static or non-static) that has been segmented from an image background. Additionally, or alternatively, a dataset output may comprise a portion or region e.g., aorta, of a heart of an individual (either static or non-static) that has been segmented from an image background and/or the remaining portion of the heart within the image.

After segmenting the image data, the segmenting software module may transmit and/or provide the one or more segmented datasets to another module within the software such as, for example, an analysis software module, and/or a modeling software module, described elsewhere herein.

### Mesh Software Module

In some cases, the segmented and/or aligned images processed by the segmenting software module may then be provided and/or transmitted to a mesh software module 306. In some cases, the mesh software module may generate one or more three-dimensional meshes of the one or more segmented and/or aligned temporal volumetric datasets of the one or more images of the patient's heart. In some instances, the three-dimensional mesh may comprise a cubic mesh. In some cases, the size of cubic mesh elements may be influenced by an extent of motion of the subject's heart, the type of partial differential equations to be solved (e.g., strain, stress, and/or diffusion), error assessment, or any combination thereof. In some cases, error assessment may comprise an estimate or prediction of the degree of error of segmentation and image realignment, described elsewhere herein. In some cases, the three-dimensional mesh generated by the mesh software module 306 may be used by other software modules (e.g., 308, 310, 312, 314, 315, 316, or any combination thereof) to calculate functional parameters, physiologic parameters, electrophysiologic parameters, electrophysiologic maps, velocity of heart structures, acceleration of heart structures, stress and/or strain tensor fields, mechanical stress and/or strain, or any combination thereof. In some instances, the mesh generated by the mesh software module may then be used as an input for the kinematic analysis software module 308 to calculate further physiologic, mechanical, electrical, or any combination thereof analysis results.

In some instances, the mesh software module may generate a mesh comprised of discrete mesh elements. In some cases, the discrete elements may comprise a cubic mesh element, tetrahedron mesh element, or any combination thereof element type. In some cases, the discrete mesh element may comprise one or more nodes, where a node may be located at a junction of two or more edges to each mesh element of the mesh generated by the one or more volumetric images of a patient's heart.

In some cases, the mesh element may comprise about 4 nodes to about 64 nodes. In some cases, the mesh element may comprise about 4 nodes to about 8 nodes, about 4 nodes to about 10 nodes, about 4 nodes to about 20 nodes, about 4 nodes to about 27 nodes, about 4 nodes to about 64 nodes, about 8 nodes to about 10 nodes, about 8 nodes to about 20 nodes, about 8 nodes to about 27 nodes, about 8 nodes to about 64 nodes, about 10 nodes to about 20 nodes, about 10 nodes to about 27 nodes, about 10 nodes to about 64 nodes, about 20 nodes to about 27 nodes, about 20 nodes to about 64 nodes, or about 27 nodes to about 64 nodes. In some cases, the mesh element may comprise about 4 nodes, about 8 nodes, about 10 nodes, about 20 nodes, about 27 nodes, or about 64 nodes. In some cases, the mesh element may comprise at least about 4 nodes, about 8 nodes, about 10 nodes, about 20 nodes, or about 27 nodes. In some cases, the mesh element may comprise at most about 8 nodes, about 10 nodes, about 20 nodes, about 27 nodes, or about 64 nodes.

### Analysis Software Module

In some embodiments, the methods, system, and/or software described herein may comprise and/or be configured to or utilize one or more analysis software modules (308, 310, 312, 314, 315, 316, or any combination thereof), as seen in FIG. 3A. An analysis software module may be configured to perform an analytic process on one or more datasets that has been received by the software. In some instances, data received by the analysis software module may be pre-processed by an input software module and/or a segmenting software module, described elsewhere herein. In some instances, an analysis software module may be configured to carry out an analysis function on the data that it receives. In some cases, the analysis function may comprise applying mathematical functions or methods (e.g., finite element analysis, convolution, cross-correlation, deconvolution, maximum intensity projection, etc.) to solve physics based mathematical models (e.g., partial differential equations prescribing laws of physics) constructed by the input data. Data received by an analysis software module, in some cases, may comprise image data, non-image data, or any combination thereof, described elsewhere herein. In some cases, the partial differential equations may pertain to the law of physics of conductance, diffusion (e.g., of a fluid), strain, stress, or any combination thereof, described elsewhere herein.

In some cases, the analysis software module may carry out a finite element analysis mathematical operation on the data that it receives, which may generate an analysis result. In some cases, the analysis result may comprise at least one equation that represents a kinematic property of a heart. In some instances, the analysis result may represent a quality and/or property of the data that correlates directly to a physiologic, anatomical, and/or electrophysiologic parameter of the heart. In some cases, finite element analysis may be applied by the analysis software module to three-dimensional mesh heart data, described elsewhere herein, to generate an analysis result comprising a mathematical representation of motion of a heart wall and/or a portion of the heart wall. In some instances, finite element analysis may be applied by the analysis software module to heart image data to generate an analysis result comprising a mathematical representation of a strain tensor of a heart wall and/or a portion of the heart wall. In some cases, finite element analysis may be applied by the analysis software module to heart image data to generate an analysis result comprising a mathematical representation of a density or flow of electrical current through heart tissue or portion of a heart tissue. In some instances, finite element analysis may be applied by the analysis software module to heart image data to generate an analysis result comprising a mathematical representation of the movement or deformation gradient of a heart wall, valve, substructure, any combination thereof, and/or any portion thereof the heart wall, valve, and/or substructure. In some cases, finite element analysis may be applied by the analysis software module to heart image data taken from an image of the heart to generate an analysis result comprising a mathematical representation of blood flow and/or perfusion through the heart or through a portion of the heart.

In some embodiments, the analysis software module may utilize non-image data, described elsewhere herein, to generate an analysis result. In some cases, the analysis software module may utilize non-image together with image data to generate an analysis result. In some instances, non-image data may provide a framework and/or inform how a finite element analysis technique may be applied to generate the analysis result.

In some instances, after generating an analysis result, the analysis software module may transmit the analysis result to another module of the software, such as, for example, a modeling software module. In some cases, the modeling software module and the analysis software module may be a single combined module of the software. In some instances, the modeling software module and the analysis software module may be separate modules of the software. In some instances, the modeling software module may display the model generated via a display module. In some cases, the display module may comprise an open-source software package, such as Para View.

### Kinematics Analysis Software Module

In some cases, an analysis software module may comprise a kinematic analysis software module, 308, as seen in FIGS. 3A-3C. In some instances, the kinematic analysis software module may take as an input, the output of the mesh module, 306, described elsewhere herein. In some cases, the kinematic analysis software module may be configured to determine a velocity and/or an acceleration of each node of the plurality of nodes of the mesh generated by the mesh module 306. In some instances, the kinematics analysis software module 308 may calculate displacement of each node of the plurality of nodes of the mesh. In some cases, kinematics analysis software module may calculate a Lagrangian displacement of each node of the plurality of nodes of the mesh.

In some cases, the velocity, acceleration, displacement, strain, or any combination thereof calculated metrics determined by the kinematics analysis software module, may then be used as in input into the pressure and flow analysis software module 312, fiber orientation analysis software module 310, energetics analysis software module 316, or any combination thereof analysis software modules, to further process the dynamic kinematic data to arrive at a physiologic analysis result, described elsewhere herein.

### Pressure and Flow Analysis Software Module

In some cases, an analysis software module may comprise a pressure and flow analysis software module 312. In some cases, the pressure and flow analysis module may be configured to receive the velocity, acceleration, displacement, strain, or any combination thereof calculated metric of the kinematics analysis software module 308, and determine a flow or pressure generated by a patient's heart wall and/or heart anatomical feature(s) on the blood within the patient's heart. In some cases, the heart anatomical features may comprise a heart valve. In some instances, the flow of blood through an atrium into a ventricle may be calculated by the pressure and flow analysis module. In some cases, flow of blood through a ventricle into supporting vasculature may be calculated by the pressure and flow analysis module. In some cases, the pressure and/or flow parameters of the blood flow within the heart may be calculated by applying Naiver stokes equations to the interaction between the heart wall and/or heat anatomical feature motion, as determined by the kinematic analysis software module 308, and the blood within the heart chambers. In some instances, blood may be considered a non-Newtonian fluid for purposes of calculating flow and pressure. In some cases, the pressure and blood flow calculated by the pressure and flow analysis module 312 may be used as an input for a kinetics analysis module 315 to calculate stress and strain of the patient's heart wall and/or heart anatomical features.

### Kinetics Analysis Software Module

In some instances, the analysis software module may comprise a kinetics analysis software module 315, configured to calculate stress and/or strain tensor matrixes for each element of the mesh generated by the mesh software module 306. In some cases, the kinetics analysis module 315 may receive as input, the pressure and flow determined by the pressure and flow analysis software module 312 to calculate the stress and/or strain tensor matrixes. In some cases, the strain and/or stress tensor matrix may comprise a matrix size of 3×3. In some cases, the strain and/or stress tensor matrixes may comprise stress and/or strain created internally within the heart wall tissue. In some cases, the kinetics analysis software module 315 may be configured to determine active stress at each node of the plurality of nodes of the each mesh element independent of another node of the plurality of the nodes of the mesh element. In some cases, the stress and/or strain tensor matrixes calculated in addition to the displacement, velocity, acceleration, or any combination thereof may then be provided as inputs to the fiber analysis software module 310.

In some cases, the kinetics analysis module 315 may iteratively improve and/or modify fiber orientation of myocardium of a patient's heart. In some cases, the iterative improvement and/or modification of the fiber orientation may be completed in tandem with the fiber analysis software module 310, described elsewhere herein.

In some cases, the kinetics analysis module 315 may compute an activation time field as a function of time and space. In some cases, the activation time field may comprise data of myocardial conductivity of a heart of the subject and/or patient.

### Fiber Analysis Software Module

In some cases, the analysis software module may comprise a fiber analysis software module 310 configured to determine the orientation of the myocardium muscle fibers of a patient's heart tissue. In some instances, the fiber analysis software module 310 may determine the orientation of at least one fiber. In some cases, the fiber analysis software module 310 may determine the orientation of a plurality of fibers. In some instances, the fiber analysis software module 310 may determine the orientation of the myocardium muscle fibers by considering the output of the displacement, velocity, acceleration, or any combination thereof, in combination with the calculated stress and/or strain tensors output of the kinetics analysis software module 315. In some cases, the displacement, velocity, acceleration, stress, strain, or any combination thereof parameters are considered by the fiber analysis software module as parameters with varying values in a spatial and temporal domains and/or dimension.

### Energetics Analysis Software Module

In some cases, the analysis software module may comprise an energetics analysis software module 316 configured to determine the metabolic activity i.e., the viability of a particular region of a patient's heart tissue. In some cases, the energetics analysis software module 316 may take as an input the displacement, velocity, acceleration, or any combination thereof output of the kinematics analysis module 308 in combination with the stress and/or strain output of the kinetics analysis software module 315. In some instances, the energetics analysis software module 316 may compute a strain energy, where the strain energy comprises the integral of the stress multiplied by the strain output of the kinematics analysis module 308. In some cases, the analysis software module 316 may compute mechanical power of a heart tissue. In some cases mechanical power may comprise the integral of the stress multiplied by the strain rate as a function of time. In some cases, healthy tissue, may be determined from a threshold value of strain energy and/or mechanical power. For example, an akinetic region of a subjects' myocardium may be associated with low values of strain energy and/or mechanical power. In some instances, dead, necrotic, and/or apoptotic cardiac myocardium may also be associated with low values of strain energy and/or mechanical power.

### Electrophysiology Analysis Software Module

In some instances, the analysis software module may comprise an electrophysiology analysis software module 314 that may be configured to determine a spatial distribution of electrical current and how it propagates through a patient's heart tissue. In some instances, the electrophysiology analysis software module 314 may receive, as an input, the output of the energetics 316 and kinetics analysis software modules 315 and output a current density plot superimposed upon the three-dimensional mesh, described elsewhere herein. In some cases, the output of the kinetics analysis software module may comprise an activation time map, described elsewhere herein. In some instances, the electrophysiology analysis software module may calculate the current density plot by solving Maxwell and/or Maxwell-like differential equations. In some instances, the electrophysiology analysis software module may determine the electrical properties of a patient's heart tissue by an inverse finite element approach. In some cases, the electrophysiology analysis software module 314 may estimate a heart's electrical conductance network that shares a relationship with a heart's purkinje fibers.

### Modeling Software Module

In some cases, the software may comprise a modeling software module. In some instances, the modeling software module may be configured to generate a model of a heart of an individual, based on data of and/or relating to the individual's heart (including image and non-image data as described elsewhere herein). In some cases, the modeling software module may generate a model (or other type of output) using an analysis result generated by the analysis software module. In some instances, the modeling software module may generate a model using one or more analysis results of the one or more analysis software modules and the mesh software module.

In some cases, the model generated by the modeling software may comprise one or more of a functional, physiologic, and/or an electrophysiologic parameter. In some instances, the model that is generated may comprises a gray scale, and/or color two or three-dimensional model of an individual's heart and/or a portion thereof. In some instances, the model of heart of the individual and/or a portion thereof may be displayed on a display of a computing device. In some cases, the model may be an interactive model that allows one or more users to manipulate the model displayed on a computer display to view different portions of the model and/or to view slices through the model. In some cases, the model may be viewed in virtual reality. In some instances, the one or more users may utilize a virtual reality headset and input devices to visualize and/or manipulate the model. In some cases, the one or more users may manipulate the model through physical touch (e.g., in the instance that the display is a touch screen display), a mouse and keyboard, or any combination thereof. In some instances, the physical touch may comprise one or more gestures that may allow a user to manipulate the model. In some cases, the one or more gestures may comprise a pinch in or pinch out to zoom in and zoom out, respectively, of the model.

In some cases, the modeling software module may utilize an artificial intelligence or machine learning algorithm configured to assist with and/or complete the one or more methods and/or calculations described by the one or more analysis modules. The artificial intelligence or machine learning algorithm may comprise a neural network. In some cases, the machine learning algorithm and/or the artificial intelligence may initially be trained on data of a population of individuals. In some cases, the data of the population of individuals may comprise image and non-image data. In some embodiments, the data of the population of individuals may comprise both normal (e.g., data of healthy individuals without disease) and abnormal data (e.g., data of individuals with disease or pre-disease states, as described elsewhere herein).

In some cases, the model may comprise and/or display one or more of a physiological, anatomical, and/or electrophysiological parameter. In some instance, the one or more physiological, anatomical, and/or electrophysiological parameters may be overlaid directly on top of the model when displayed and/or associated with the model or section and/or region of the model, e.g., the physiological, anatomical, and/or electrophysiological parameters may be revealed when a mouse or cursor is held over the model or a section of the model by one or more users.

In some embodiments, the modeling software module may generate a model that comprises in vivo spatial and temporal measurements of a full strain tensor field. In some instances, the strain tensor field may comprise up to six components. In some cases, the stress tensor field may comprise up to six components. In some instances, the modeling software module may generate a model that may comprise in vivo spatial measurements of stored energy, e.g., stored stress and/or residual stress of the heart wall, particularly in the ventricle (LV) wall. In some cases, the modeling software module may generate a model comprising spatial and temporal force, stress and energy fields generated by myocardium contraction. In some instances, models generated by the software and methods described herein may present and/or display the coupling of electrical stimuli and muscle contraction throughout a full cardiac cycle. In some cases, the modeling software module may generate a model comprising in vivo measurements of the spatial and temporal electrical potential difference field of a heart non-invasively, enabling a determination of a heart tissue conductance tensor field. In some instances, the modeling software module may generate a model comprising myocardium in vivo electrical activities as a function of time and/or in three-dimensional space. In some cases, a model generated by the modeling software module may comprise a voltage map and/or an activation time map of an individual's entire heart and/or a portion thereof. In some instances, a model generated by the modeling software module may comprise at least one vector representing a current flow through an individual's heart or a portion of thereof. In some cases, a model generated by the modeling software module may comprise an individual's heart wall thickness measurements or a thickness of a portion of the heart wall. In some instances, a model generated by the modeling software module may comprise information related to at least one valve of the heart including, for example, valve movement dynamics and/or valve competency. In some cases, a model generated by the modeling software module may comprise measurements of the flow of blood and/or the diffusion of blood through the heart, heart tissues, or a portion or region thereof. In some instances, a model generated by the modeling software module may comprise oxygen metabolism measurements of at least a portion of the heart of the individual. In some cases, a model generated by the modeling software module may comprise measurements of heart tissue perfusion, e.g., tissue perfusion of heart myocardium. In some instances, a model generated by the modeling software module may comprise a measure of scar tissue formation over a portion of myocardium of a heart. In some cases the model, when displayed may comprise range based visual indicators of each measure or parameter of the model, described elsewhere herein. In some cases, the visual indicator may comprise an overlay of a gray scale map, color map, heat map, or any combination thereof, where the overlay is spatially superimposed over the three-dimension model of a heart. In some instances, the displayed overlay may be correlated to a legend simultaneously displayed that may inform one or more users of the numeric value or categorical description of a particular measure or parameter, described elsewhere herein.

In some embodiments, certain data may not be associated with a model that is displayed but rather is outputted in other formats such as, for example, as a raw numerical value. In some instances, the data not associate with the model may comprise cardiac efficiency values. In some cases, the data not associated with the model may comprise quantitative values associated with one or more of the American heart association (AHA) cardiac segments. In some cases, the model and/or data that is not associated with the model may be outputted as text, data (e.g., “.txt”, “.dat”) for future review, storage, and/or HIPPA compliant transfer of data between patient and provider and/or between two different providers.

It should be understood that any of the software modules may be combined into a single module or carried out by fewer modules. The software modules described herein provide an exemplary way to carry out the methods and/or software algorithm described herein and those having skill in the art will understand that modules having different names or different functions are equally suitable for use with the software described herein. One or more software modules described herein may accept and/or receive as an input one or more outputted results of calculations and/or processed data. The one or more software modules for purposes of clarity have been explained independent of one another, however, one of ordinary skill in the art would readily realize and understand that the software described herein may comprise one or more of such software modules to provide one or more analysis results as described elsewhere herein.

### Therapeutic Optimization Module

In some cases, the methods, system, and/or software may comprise a therapeutic optimization module 317, as seen in FIG. 3B. In some cases, the therapeutic optimization module may take as an input the mesh 306, kinematics 308, fiber 310, electrophysiology 314, pressure and flow 312, kinetics 315, or any combination thereof analysis modules' outputs. In some instances, the therapeutic optimization module may provide and/or guide surgical intervention of a subject's heart. In some instances, the therapeutic optimization module may output a three-dimensional map and recommendation of regions of a patient's heart that would benefit from a gel injection. In some instances, the gel-injection may be configured to reduce local stress on the region of the failing heart tissue and/or prevent further dilation of an affected ventricle and/or atrium of the subject's heart. In some cases, the therapeutic optimization module output may guide the design of material properties and/or stiffness of heart-based implants. In some cases, a physician may implant a rigid structure over one or more regions of a subject's heart to constrain the one or more regions of the subject's heart prevent dilation caused by heart failure. In this instance, the therapeutic optimization module may be used to determine parameters of the implant based on the analysis and output provided by the inputs, described above.

In some cases, the therapeutic optimization module 317, may output one or more therapeutic guidance parameters. In some cases, the one or more therapeutic guidance parameters may be displayed as a three-dimensional model of therapeutic guidance parameters superimposed upon a three-dimensional volumetric representation of a subject's heart. In some cases, co-registration between the two models may provide a view for a health care provider and/or physician to relate the therapeutic guidance parameter to heart anatomy.

### Active Growth Remodeling Module

In some cases, the methods, system, and/or software may comprise an active growth remodeling module 319, as seen in FIG. 3C. In some instances, the active growth remodeling module 319 may take as an input the mesh 306, kinematics 308, fiber 310, pressure and flow 312, kinetics 315, energetics 316, or any combination thereof analysis modules' outputs. In some instances, the active growth remodeling module may be configured to determine the effect and/or result of clinical intervention to treat heart disease and/or condition of a subject. The active growth remodeling module may be configured to monitor the resulting effect of providing a gel based injection treatment for a patient's heart failure. The active growth remodeling module may analyze both in-vivo and/or in-vitro datasets produced by one or more of the aforementioned modules, described elsewhere herein. In some cases, the in-vivo and/or in-vitro datasets may comprise one or more longitudinal datasets of a subject's MRI scan over a period time.

In some cases, the output of the active growth remodeling module may comprise one or more growth rate parameters. In some instances, the one or more growth rate parameters may comprise a growth rate of a patient's myocardium in both spatial and temporal domains. In some cases, the one or more growth rate parameters may be modeled into a three-dimensional growth rate parameters super-imposed over three-dimensional volumetric model of a subject's heart, described elsewhere herein. In some cases, co-registration between the two models may provide a view for a health care provider and/or physician to relate the therapeutic guidance parameter to heart anatomy.

### Computer-Implemented Method

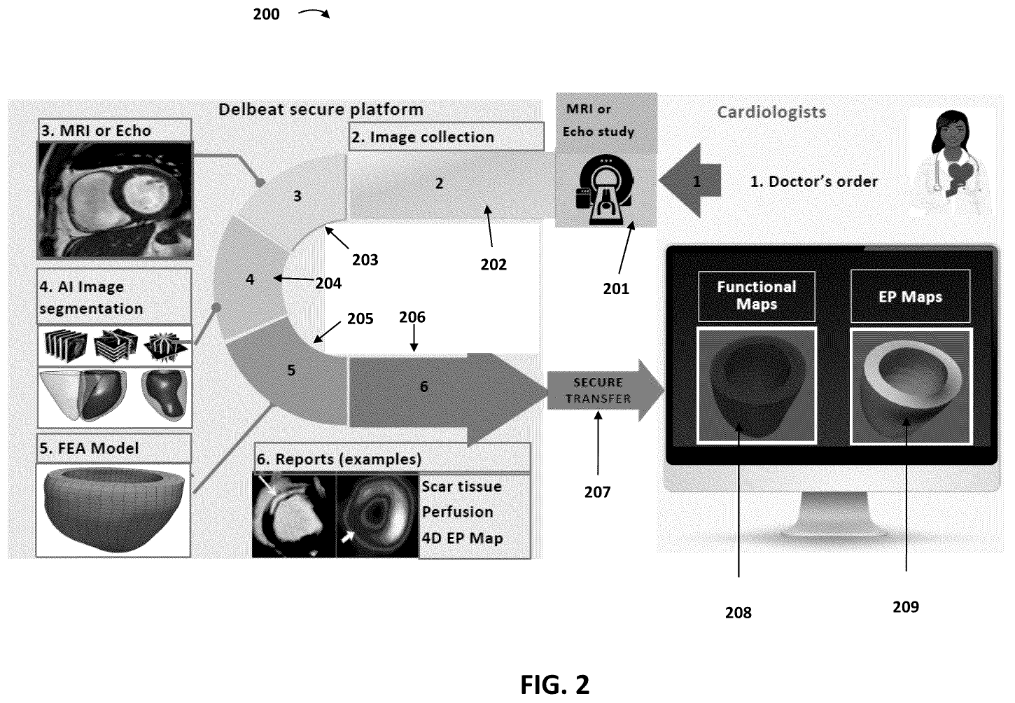

FIG. 2 shows a schematic of an exemplary workflow that may comprise a computer-implemented method 200 described herein. In step 201, a patient undergoes an imaging procedure that may capture and/or record the heart of the individual in image form. As shown in FIG. 2, the imaging procedure may comprise an MRI, DENSE, tag-MR, SPAMM, 4D-flow, echocardiogram, CTA, or any combination thereof imaging modality. In some cases, the MRI may comprise a CINE MRI. In some instances, the echocardiogram may comprise a two-dimensional, three-dimensional, four-dimensional, or any combination thereof echocardiogram. In some cases, the imaging procedures may comprise CT, MRI, CT angiography, time-resolved CTA, X-ray, traditional angiogram, or any combination thereof. In some cases, the data may comprise an MRI sequence determined from compressed sensing or accelerated acquisition. In a step 202, the one or more images generated by the imaging procedure of step 201 may be received by an input software module, described elsewhere herein. In some cases, the one or more images may be transferred to the input software module 202 via an online secure HIPPA compliant portal and/or cloud-based server over a network. In some instances, the one or more images may be transferred to the input software module 202 via Bluetooth or Wi-Fi communication protocols. In some cases, the one or more images may be transferred to the image input software module through a wired electrical communication. The images may be transferred to the input software module through a non-transitory storage medium e.g., a flash memory thumb drive, CD-ROM, DVD-ROM, etc. An input software module 202 may be configured to receive and process data transmitted to a computing device. In some cases, data received by the input software module may be associated with and/or represent a heart of an individual. In some instances, the data received by the input software module may comprises one or more images of a heart or a portion of a heart. In some cases, data that is received may comprise non-static images such as, for example, images that show cardiac motion and/or blood flow. Non-limiting examples of imaging procedures that may produce one or more images that show cardiac motion may comprise echocardiogram, time-resolved CTA, MRIs, CT, or any combination thereof imaging procedures. In some cases, the echocardiogram may comprise a four-dimensional echocardiogram. In some instances, the MRI may comprise a CINE MRI. As used herein an “image” includes a single image or a collection of images together (e.g. a video or motion capture). “Static” refers to images that do not capture cardiac motion (i.e. kinetics). “Non-static” refers to images that show cardiac motion (i.e. kinetics).

Data received by the input software module 202, in some cases, may comprise non-image data specific to the individual such as ECG, medical record data, and/or demographic data.

In some instances, non-limiting examples of data received by the input software module may comprise: the individual's demographic data, medical record data (including electronic medical record data), ECG data, two-dimensional, three-dimensional, and/or four-dimensional echocardiogram data, MRI data, CINE MRI data, CT data, CT angiography data, time-resolved CTA, X-ray data, traditional angiogram data, or any combination thereof data. Data that is received may be complete or partial with respect to the data itself. In some cases, data received may be associated with, relate to, and/or show the heart of an individual who is being evaluated for cardiac disease. In some cases, data received may comprise data from individuals of a population that are not being immediately evaluated using the method and/or software. In some cases, the data received may comprise data retrospectively analyzed (i.e., was acquired at a time point earlier than the analysis)

Data may be received by the input software module 202 through any known means of communication including through wired or wireless communication with another computing device, server, cloud, and/or any device capable of transmitting data to the computing device that is running the input software module.

In some cases, data received by the input software module may also be processed by the input software module. For example, in some instances, data received may be identified (e.g., with respect to content or type of data) by the input software module and assigned a certain value based on its identification which further determines how the received data is handled by the software. For example, an ECG that is received by the input software module, in some cases, may be identified as an ECG rather than an image of the heart and may be transmitted to a different software module than a received image of a heart. In some instances static images of the heart may be identified and transmitted to a different software module than non-static images of the heart. In some embodiments, the input software module may clean, in an optional step 203, the received data with respect to, for example, associated metadata and labels. In some cases, the cleaning process may comprise denoising the data to remove image artifact noise and to increase signal to noise ratios of relevant signals and/or regions of the data to be analyzed.

In an optional step 203, an input software module 202 may further processes the data that is received as described above. In some cases, the further processing may comprise: (1) de-identifying the patient information from the data, (2) reordering data, (3) re-arranging data, (4) filtering data, and (5) superimposing data.

In some cases, a segmenting software module 204 may segment the one or more images received in by the input software module 202 and optionally processed in step 203. In some instances, the segmenting software module 204 may be configured to segment one or more images of a heart received. In some cases, segmenting may comprise the process of identifying and separating the one or more image data into different data collections. For example, a segmenting software module, described elsewhere herein, may be configured to separate image data determined to be relevant (i.e., signal) to the analysis of the individual's heart by the software from background that would otherwise be considered noise and/or does not influence the analysis of the individual's heart by the software. In some cases, the segmenting software module may identify a heart within an image and separate the heart image data from background elements in the image. In some instances, segmenting may be applied to both static and/or non-static image components. Segmenting can further be applied to different anatomical components of a heart. For example, an image may be segmented to separate (or isolate) the aorta from the heart from the macroscopic structure of the heart within an image. In some cases, the segmentation software module may be configured to segment and/or separate heart wall tissue (i.e., myocardium) from the heart blood pool and/or the heart pericardium.

In some cases, the segmenting software module may comprise and/or utilizes an artificial intelligence or machine learning algorithm to segment the one or more images received. The artificial intelligence and/or machine learning algorithm may comprise a neural network and/or a convolutional neural network configured to use computer vision to analyze the one or more received images and/or determine where to segment the one or more images into one or more data collections. In some cases, the machine learning algorithm and/or the neural network may be initially trained on a population dataset. In some instances, the population dataset may comprise image data, non-image data, or any combination thereof data. In some instances, the population data may comprise normal (i.e., data of healthy individuals without a disease or pre-disease state), abnormal data (i.e., data of individuals with a disease or pre-disease states), or any combination thereof data.

In some cases, the segmenting software module 204 may output one or more data collections of image data that has been separated from other data within an image. For example, an output of the one or more collections of data may comprise a heart of an individual (either static or non-static) segmented from the image background surrounding the heart. In some cases, the output one or more data collections may comprise a portion of a heart of an individual e.g., the aorta (either static or non-static), that has been separated and/or segmented from an image background and/or the remaining portion of the heart within the image.

After segmenting the image data, the segmenting software module 204 may transmit the one or more data collections to another module of the software such as, for example, an analysis software module 205, and/or a modeling software module 206.

In some cases, the analysis software module 205, may receive an image that has been segmented in by the segmentation software module 204 into one or more data collections. The analysis software module 205 may analyze the one or more data collections of the segmented image and generate one or more analysis results. In some cases, the analysis software module 205 may be configured to solve physics-based partial and/or ordinary differentiation equations that describe: heart wall motion, strain and/or stress; blood flow; heart valve dynamics, stress and/or strain; and heart tissue conductance. In some cases, the physics-based partial and/or ordinary differentiation equations may be solved by artificial intelligence approaches, numerical methods, finite element analysis, or any combination thereof. In some cases, the analysis software module may be configured to perform an analytic process on the one or more data collections received by the software. In some instances, the data received by the analysis software module may be pre-processed by an input software module and/or a segmenting software module. In some cases, the analysis software module may be configured to carry out an analysis function on one or more data collections it receives, which may comprise applying certain mathematical functions to the one or more data collections. Data received by an analysis software module, in some cases, may comprise: image data, non-image data, or any combination thereof.

In some cases, an analysis software module may conduct finite element analysis on the one or more data collections, which generates one or more analysis result. In some instances, the analysis result generated by the analysis software module may comprises at least one equation that represents a kinematic property of the heart. In some instances, the one or more analysis result may comprise a quality or property of the one or more data collections that correlates directly to an anatomical, physiologic, and/or electrophysiologic parameter of the heart.

In some cases, finite element analysis may be conducted by the analysis software module on heart image data to generate an analysis result comprising a mathematical representation of motion of a heart wall or portion of a heart wall. In some instances, finite element analysis may be conducted by the analysis software module on heart image data taken to generate an analysis result comprising a mathematical representation of a constitutive material properties of a heart wall or a portion of a heart wall. In some cases, finite element analysis may be conducted by the analysis software module on heart image data to generate an analysis result comprising a mathematical representation of a density or flow of electrical current through a heart or portion of a heart. In some instances, finite element analysis may be conducted by the analysis software module on heart image data to generate an analysis result comprising a mathematical representation of the movement or constitutive material properties of one or more valves of the heart or a portion thereof the one or more valves of the heart. In some cases, finite element analysis may be conducted by the analysis software module on heart image data to generate an analysis result that may comprise a mathematical representation of blood flow and/or diffusion through the heart and/or heart tissues.

In some cases, the analysis software module may utilize non-image data, described elsewhere herein, to generate an analysis result. In some instances, the analysis software module may utilize non-image data together with image data to generate an analysis result. For example, non-image data may provide information regarding finite element analysis initial conditions, boundary conditions, model weight parameters, or any combination thereof finite element analysis parameters.

After generating the analysis result, the analysis software module may transmit the analysis result to another module within the software such as, for example, a modeling software module 206. It should, however, be understood that a modeling software module and analysis software module as described herein may be combined into a single module and/or be separate individual modules of the software.

In some cases, a modeling software module 206 may generate a model (or other output) based on the analysis result that is generated by the analysis software module 205. The modeling software module 206 may be configured to generate a model of a heart of an individual based on data of and/or relating to the individual's heart (including image and non-image data described elsewhere herein). In some cases, the modeling software module 206 may generate a model (or other type of output) using the analysis result generated by the analysis software module (or alternatively, an analysis result that is generated by a module that combines the function of the analysis and the modeling software modules). In some cases, the analysis result may be plotted on a two-dimensional and/or a three-dimensional plot to be viewed by one or more users of the software. In some instances, the two-dimensional and/or three-dimensional analysis results may be superimposed and co-registered to a three-dimensional model (i.e., three-dimensional mesh, described elsewhere herein) of a heart.

In some cases, the model generated by the modeling software may comprise one or more of a functional, physiologic, and/or an electrophysiologic parameter. In some cases, a model may comprise a black and white and/or a color two or three-dimensional model of the heart of the individual and/or a portion of the heart of the individual. In some instances, the heart of the individual may be displayed on a display of a computing device. In some embodiments, the model may comprise an interactive model that allows a user to manipulate the model within a computer display in order to view different portions of the model or to view slices through the model. In some instances, slices through the model may comprise slices that do not intersect the entirety of the model. In some cases, the slices through the model may comprise slices that intersect the entirety of the model. In some instances, the slices through the model may comprise slices parallel with a planar axis formed between at least 2 dimensions (e.g., x-y, y-z, or z-x plane) of a three-dimensional space of the model.

In some instances, the model may be viewable and manipulated by one or more users of the software through in an augmented reality (AR), virtual reality (VR), and/or metaverse environments. In some cases, the software, methods, and systems described here may recognize gestures and/or movements of user input devices (AR or VR joysticks and/or controllers) that may be configured to allow one or more users to interact with and manipulate the model.

In some cases, the modeling software module may comprise and/or utilize an artificial intelligence and/or machine learning algorithm. The artificial intelligence and/or machine learning algorithm may comprise a neural network. In some instances, the machine learning algorithm and/or the artificial intelligence may be initially trained on population data. In some cases, the population data may comprise image and/or non-image data. In some instances, the population data may comprise both normal (i.e., healthy) and abnormal data (i.e., indicating disease or pre-disease states).

In some cases, a model may comprise and/or display one or more of a functional, physiological, and/or an electrophysiological parameters. In some instances, the one or more functional, physiological, and/or electrophysiological parameters may be overlaid on top of the model and/or otherwise associated with the model or a section of the model (e.g., data of the one or more physiological and/or electrophysiological parameters may be displayed when a cursor is held over the model or a section of the model). In some cases, the cursor may be displaced and/or moved across a display in response to movement of an input device. In some instances, the input device may comprise a mouse, trackpad, joystick, user finger or hand, or any combination thereof. In some cases, the input device may comprise an AR and/or VR input device (e.g., joystick with built in gyroscope, accelerometer, magnetometer, and/or positioning sensors).

In some cases, the modeling software module may generate a model that comprises and/or displays in vivo spatial and/or temporal measurements of a strain and/or stress tensor field. In some instances, the strain tensor field comprises a full strain tensor field. In some cases, the strain tensor field may comprise up to 1 strain, up to 2 strains, up to 3 strains, up to 4 strains, up to 5 strains, or up to 6 strains. In some instances, the stress tensors may comprise up to 1 stresses, up to 2 stresses, up to 3 stresses, up to 4 stresses, up to 5 stresses, or up to 6 stresses. In some cases each stress or strain component of the stress and/or strain tensor may comprise active and/or passive components. In some instances, the stress and/or strain tensors may be determined in a fiber, cross fiber, sheet, or any combination thereof directions. In some embodiments, the modeling software module may generate a model that comprise and/or displays in vivo spatial measurements of stored energy, i.e., a stored stress or residual stress of an individual's heart wall and/or left ventricle (LV) wall. In some cases, the modeling software module may generate a model that comprises and/or displays spatial and temporal force, e.g., stress and energy fields generated by myocardium contraction(s). In some instances, models generated by the software, system, and/or methods, described elsewhere herein, may present and/or display a coupling of electrical stimuli and muscle contraction throughout a full cardiac cycle. In some cases, the coupling of electrical stimuli and muscle contractions may be displayed as an overlay of data spatially and/or temporally superimposed on a structural model of an individual's heart. In some embodiments, the modeling software module may generate a model that comprises and/or displays in vivo measurement of the spatial and temporal electrical potential difference field non-invasively. In some cases, the measurement of the spatial and temporal electrical potential differences may permit determination of a tissue conductance tensor field. In some cases, the modeling software module may generate a model that comprises and/or displays myocardium in vivo electrical activities as a function of time and space (i.e., a four-dimensional measurements). In some instances, a model generated by the modeling software module may display and/or present an electrical current density map of an entire heart or a portion of a heart of an individual. In some cases, a model generated by the modeling software module may display and/or present at least one vector representing a current flow through the heart or a portion of the heart of the individual. In some instances, a model generated by the modeling software module may display and/or present a wall thickness of at least one wall of a heart of an individual. In some cases, a model generated by the modeling software module may display and/or present information related to at least one valve of the heart including, for example, valve movement dynamics and valve competency. In some instances, a model generated by the modeling software module may display and/or present a flow of blood through a heart and/or a portion of heart tissue (e.g., myocardium). In some cases, a model generated by the modeling software module may display and/or present an oxygen and/or fatty acid metabolism measurement of at least a portion of the heart of the individual. In some instances, a model generated by the modeling software module described herein may display and/or present a level of tissue perfusion of an individual's heart myocardium tissue. In some cases, a model generated by the modeling software module may display and/or present scar tissue identified superimposed over a portion of heart myocardium of an individual. In some cases, a model generated by the modeling software module may display and/or present data regarding spatial and/or temporal tissue mechanical efficiency.

In some cases, some data may be not associated with a model that is displayed but rather is outputted in other formats such as, for example, as a raw numerical value.

In some instances, a communication interface 207, such as a secure communication, may transmits the output of the modeling software module to a display device for display (208, 209). The display device may be a component of the same computing device that is running the software described herein or may be a remotely located display. In some cases, the display device may comprise a remote display device, personal computing device, laptop computing device, tablet, smartphone, AR display, VR display, or any combination thereof. In some cases, one or more users may be able to view a model and/or related non-model data (e.g., raw numeric values) from a web-portal and/or from a web-browser window. In some instances, web-browser may point to a URL of a web-based server where a model and/or related non-model data may be viewed by one or more users. In some instances, the model and/or related non-model may be downloaded from the web-based server and/or portal to a local device (i.e., personal computer, laptop computer, tablet, smartphone, etc.). In some cases, the model and/or related non-model downloaded data may be visualized using Para View open-source software or other open-source software configured to display or view a model of an individual's heart.

In some instances, an output of the modeling software module may comprise a model in the form of a functional map 208 and/or in the form of an electrophysiological parameter (EP) map 209. In these exemplary models 208 and 209, one or more functional, physiological, and/or an electrophysiological parameters may be either overlaid on the model and/or otherwise associated with the model or a section of the model (e.g., data is revealed when a mouse or cursor of a user input device, described elsewhere herein, is held over the model or a section of the model).

It should be understood that any of the software modules may be combined into a single module or carried out by fewer modules. The software modules described may provide an exemplary way to carry out the software algorithm described herein and those having skill in the art will understand that modules having different names or different functions are equally suitable for use with the software described herein.

It should be further understood that both the methods and software described herein can utilize one or more computers. A computer can include a monitor or other graphical interface for displaying data, results, models, or other outputs. A computer can also include means for data or information input. A computer can include a processing unit and fixed or removable media or a combination thereof. A computer can be accessed by a user in physical proximity to a computer, for example via a keyboard and/or mouse, or by a user that does not necessarily have access to the physical computer through a communication medium such as a wired or wireless communication signal carrier wave. In some cases, a computer can be connected to a server or other communication device for relaying information from a user to a computer or from a computer to a user. In some cases, the user can store data or information obtained from a computer through a communication medium on media, such as a removable medium. It is envisioned that data relating to the computer-implemented methods described herein can be transmitted over such networks or connections for reception and/or review by a party. The receiving party and/or user can be, but is not limited to, an individual, a health care provider or a health care manager. In some cases, a computer-readable medium includes a medium suitable for transmission of an output of a software or computer-implemented method as described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. One or more embodiments and/or software modules, algorithms, machine learning models, methods, or any combination thereof, as described elsewhere herein, may be combined together into a single embodiment. It should be understood that various alternatives to the embodiments of the invention described herein may be employed in practicing the invention. It is intended that the following claims define the scope of the invention and that methods and structures within the scope of these claims and their equivalents be covered thereby.

Although the above steps show each of the methods or sets of operations in accordance with embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial. One or more of the steps of each of the methods or sets of operations may be performed with circuitry as described herein, for example, one or more of the processor or logic circuitry such as programmable array logic for a field programmable gate array. The circuitry may be programmed to provide one or more of the steps of each of the methods or sets of operations and the program may comprise program instructions stored on a computer readable memory or programmed steps of the logic circuitry such as the programmable array logic or the field programmable gate array, for example.

Additional exemplary embodiments will be further described with reference to the following examples; however, these exemplary embodiments are not limited to such examples.

### EXAMPLES

### Example 1: Using Software to Determine Heart Disease State of an Individual From Images of an Individual's Heart

An individual suspected of having heart disease and/or a heart disorder will come into a cardiology clinic and describe his/her symptoms to a physician and/or medical personnel that will then order imaging of the individual's heart. The individual will then undergo spatial-temporal imaging (e.g., three-dimensional echocardiogram, MRI, Tag-MR, CINE MRI, or any combination thereof imaging) of their heart generating one or more images. The one or more images generated by the imaging system will be transmitted to an input software module through wired or wireless communication with the imaging system. Next the data will be further cleaned (e.g., denoising) and processed with an input software module. After cleaning and processing with the input software module, a software segmentation module will then segment the one or more images outputted by the input software module to segment the individual's heart from the background of the other tissue and/or organ systems of the individual into a data collection. The heart will then be further segmented to one or more additional data collections comprising one or more anatomical features (e.g., ventricle, atrium, heart wall, heart valve, etc.) of the heart for further analysis. The heart and/or the one more additional data collections will then be transported to an analysis software module that will analyze each of the one or more data collections producing one or more analysis results. The one or more analysis results will include, e.g., a finite element analysis to represent density or flow of electrical current through the individual's heart, movement and/or constitutive material properties of one or more valves of the individual's heart, blood flow and/or diffusion of blood through the heart and/or heart tissues of the individual, or any combination thereof analysis results. The analysis results will then be transmitted to a modeling software module that will combine the analysis results into a model. The model will then be displayed to one or more users via a personal computer, smartphone, web-browser, tablet, laptop computer, cloud-based computing platform, AR environment, VR environment, metaverse, or any combination thereof interactive display. The model will be interactive, such that the one or more users will be able to interact with the model through interaction with a cursor or pointer controlled by a user input device (e.g., mouse, mouse pad, joystick, etc.) bisect and/or slice the model and determine the analysis results associated with a spatial position on the model. The model and/or related raw numerical values of the analysis result may be exported for offline analysis or filing in the individual's medical records and/or importing into an electronic medical record system.

1.-77. (canceled)

78. A computer-implemented method for generating a model of a heart of an individual, the method comprising:
(a) receiving or obtaining, with an input software module, an image of the heart of the individual;
(b) segmenting, with a segmenting software module, the image of the heart, thereby generating at least one image segment;
(c) applying, using an analysis software module, one or more differential equations to the at least one image segment, thereby generating at least one analysis result; and
(d) generating, using a modeling software module, the model of the heart of the individual using the analysis result of (c).

79. The method of claim 78, wherein the analysis result comprises at least one functional feature, at least one electrophysiological feature, or any combination thereof features.

80. The method of claim 79, wherein the functional feature comprises a ventricular gauge pressure of a right and left ventricle.

81. The method of claim 79, wherein the functional feature comprises a wall thickness of a chamber of the heart.

82. The method of claim 79, wherein the functional feature comprises a wall motion of at least a portion of the heart.

83. The method of claim 79, wherein the electrophysiological feature comprises an electrical property of a tissue of the heart, and wherein the electrical property of the tissue of the heart comprises an activation map, voltage map, or any combination thereof.

84. The method of claim 79, wherein the electrical property comprises at least one current vector.

85. The method of claim 78, wherein the image of the heart is an echocardiogram image.

86. The method of claim 85, wherein the echocardiogram comprises a 3D echocardiogram.

87. The method of claim 78, comprising generating a growth remodeling parameter model with the analysis results of (c) longitudinally over a period of time.

88. A non-transitory computer readable medium that includes software that uses artificial intelligence to model a heart of an individual, wherein the software causes a processor to:
(a) receive or obtain an image of the heart of an individual;
(b) segment the image of the heart, thereby generating at least one image segment;
(c) apply a series of numerical techniques to the at least one image segment, thereby generating an analysis result; and
(d) generate, a model with at least one functional feature, at least one electrophysiological feature, or a combination thereof, of the heart of the individual using the analysis result of (c).

89. The non-transitory computer readable medium of claim 88, wherein the functional feature comprises a ventricular gauge pressure.

90. The non-transitory computer readable medium of claim 88, wherein the functional feature comprises a wall thickness of a chamber of the heart.

91. The non-transitory computer readable medium of claim 88, wherein the electrophysiological feature comprises an electrical property of a tissue of the heart, and wherein the electrical property of the tissue of the heart comprises an activation map, voltage map, or any combination thereof.

92. The non-transitory computer readable medium of claim 91, wherein the electrical property comprises at least one current vector.

93. The non-transitory computer readable medium of claim 88, wherein the image of the heart comprises an MRI image, and wherein the MRI comprises CINE MRI, MRI based techniques, or any combination thereof.

94. The non-transitory computer readable medium of claim 93, wherein the MRI based techniques comprise DENSE, tag-MR, SPAMM, or any combination thereof.

95. The non-transitory computer readable medium of claim 88, wherein the image of the heart is an echocardiogram image.

96. The non-transitory computer readable medium of claim 95, wherein the echocardiogram comprises a 3D echocardiogram.

97. A method for determining a material property of a region of a heart of an individual, the method comprising:
(a) receiving or obtaining an image of the heart of an individual;
(b) segmenting the image of the heart, thereby generating at least one image segment;
(c) applying one or more differential equations to the at least one image segment, thereby generating at least one analysis result, wherein the at least one analysis result comprises a functional parameter, an electrophysiological parameter, or a combination thereof; and
(d) determining a material property of at least one region of the heart of the individual by the at least one analysis result.
